(19) United States
(12) Patent Application Publication
(10) Pub. No.: US 2003/0208247 A1
(43) Pub. Date: Nov. 6, 2003

(54) Implantable stimulation lead with tissue in-growth anchor

(76) Inventors: Michele Spinelli, Milano (IT); Martin T. Gerber, Maple Grove, MN (US); John M. Swoyer, Andover, MN (US); Gianluca Giardiello, Milano (IT); Andrea Arduini, Padova (IT)

Correspondence Address: MEDTRONIC, INC., 710 MEDTRONIC PARKWAY NE, MS-LC340, MINNEAPOLIS, MN, 55432-5604, US

(21) Appl. No.: 10/015,503

(22) Filed: Dec. 11, 2001

Related U.S. Application Data
(60) Provisional application No. 60/325,904, filed on Sep. 28, 2001.

(51) Int. Cl. A61N 1/05

(52) U.S. Cl. 607/51

(57) ABSTRACT

An implantable neurological stimulation system has an in-growth anchor coupleable to an implantable stimulation lead that configured for anchoring the stimulation lead to tissue. The stimulation lead is coupled to an implantable neurostimulator, and the in-growth anchor is positioned proximal to at least one electrode. Many embodiments of the implantable neurological stimulation system with in-growth anchor and its methods of operation are possible.

### RELATED APPLICATIONS

This application claims the benefit of provisional application, U.S. Ser. No. 60/325,904, filed on Sep. 28, 2001. This disclosure is related to the following co-pending applications entitled “Minimally Invasive Surgical Techniques For Implanting Devices That Deliver Stimulation To The Nervous System” by inventors Gerber et al. (application Ser. No. 09/489,544; filed Jan. 31, 2000) and “Minimally Invasive Apparatus For Implanting A Sacral Stimulation Lead” by inventors Mamo et al. (application Ser. No. 09/827,740, filed Apr. 7, 2001), which are not admitted as prior art with respect to the present disclosure by its mention in this cross reference section.

### FIELD OF THE INVENTION

This invention relates to a medical device and more particularly to a neurological stimulation lead anchor that can be implanted in a human body.

### BACKGROUND OF THE INVENTION

The medical device industry produces a wide variety of electronic and mechanical devices such as neurological stimulators, therapeutic substance infusion pumps, pacemakers and defibrillators for treating patient medical conditions such as pain, movement disorders, functional disorders, spastisity, cancer, and cardiac disorders. Medical devices ca-i be configured to be surgically implanted or connected externally to the patient receiving treatment and can be used either alone or in combination with pharmaceutical therapies and surgery to treat patient medical conditions. For certain medical conditions, medical devices provide the best and sometimes the only therapy to restore an individual to a more healthful condition and a fuller life. One type of medical device is an implantable neurological stimulation system that typically includes a neurostimulator, an electrical stimulation lead, and an extension such as shown in Medtronic, Inc. brochure “Implantable Neurostimulation System” (1998). An implantable neurological stimulation system delivers electrical pulses to tissue such as neurological tissue or muscle to treat a medical condition such as pelvic floor disorders.

Pelvic floor disorders adversely affect the health and quality of life of millions of people. Pelvic floor disorders include urinary control disorders such as urge incontinency, urge frequency, voiding efficiency, fecal control disorders, sexual dysfunction, and pelvic pain. Individuals with urinary control disorders often face debilitating challenges in their everyday lives. These individuals can be preoccupied with trips to the bathroom, fears of embarrassment, and sleepless nights. Some suffers become so anxious that they become isolated and depressed. Pelvic floor disorders can be treated with a variety of therapeutic options such as behavior modification including biofeedback, pharmacological treatment, mechanical intervention such as self-catheterization, physical appliances such as diapers, and surgical intervention. Surgical treatments are the most invasive and are often considered after other therapies have proven ineffective

One surgical technique to treat urinary control disorders is the implantable InterStim® therapy, available from Medtronic, Inc., that applies mild electrical stimulation to the sacral nerves in the lower region of the spine to influence behavior of structures such as the bladder, sphincter and pelvic floor muscles. Generally, implantation of InterStim therapy involves surgically implanting a stimulation lead near the sacral nerves. The stimulation lead is a very small, insulated electrical conductor with electrical stimulation contacts on the distal end placed near the sacral nerves and an electrical connector on the opposite proximal end of the lead. The lead electrical connector is typically connected to a small extension, and the extension is connected to a small neurostimulator that operates similar to a cardiac pacemaker by delivering occasional small electrical pulses that sometimes create a tingling sensation felt by the patient. The stimulation lead, lead extension, and neurostimulator are all implanted in the patient in a manner that is typically not perceptible by others. InterStim therapy can improve the condition of a pelvic floor disorder patient and allow the patient to lead a full life, and the therapy is nondestructive and reversible.

Previous surgical methods to implant a neurostimulation lead in a patient's sacrum to treat pelvic floor disorders have been invasive by requiring a large sacral incision in a procedure known as dissection. Dissection involves making a midline incision over the sacrum from a little below S4 up to S1 that in an adult ranges from about 7.62 cm (3.0 inches) to 12.7 cm (5.0 inches). After the incision is made, the fascia lateral to the midline is cleaned off and divided in the direction of the incision approximately one finger width lateral to the midline. Next, the paraspinal muscle fibers are split and sharply retracted. Once the muscle fibers are retracted, the sacral foramen is exposed while preserving the periosteum. Next, the desired foramen is located by observing anatomical landmarks and palpating for a marble-board-like depression. Once the desired foramen is located, another small incision is made over the desired foramen that is large enough to allow insertion of the stimulation lead. The stimulation lead is inserted through the incision. An example of the previous surgical method to implant a neurostimulation lead is described in Medtronic, “InterStim® Therapy Sacral Nerve Stimulation For Urinary Control Therapy Reference Guide,” Section 5 InterStim Device Implantation Procedure, pp. 51-52 (1999).

For the foregoing reasons, there is a need a stimulation lead anchor that does not require a surgical procedure to attached the anchor to tissue.

### BRIEF SUMMARY OF THE INVENTION

The implantable neurological stimulation system has an in-growth anchor coupleable to an implantable stimulation lead that is configured for anchoring the stimulation lead to tissue. The in-growth anchor is positioned proximal to and not in contact with at least one electrode. The stimulation lead is coupled to an implantable neurostimulator. Many embodiments of the implantable neurological stimulation system with in-growth anchor and its methods of operation are possible.

### BRIEF DESCRIPTION OF THE DRAWINGS

FIG. 1 (prior art) shows a patient undergoing a sacral stimulation procedure;

FIG. 2 shows a neurological stimulation system environment embodiment;

FIG. 3 shows a neurological stimulation lead embodiment;

FIGS. 4a-4b show in-growth anchor material embodiments;

FIG. 5 shows a schematic of an implanted sacral stimulation lead embodiment;

FIG. 6 shows an anatomical drawing of a sacrum embodiment; and,

FIG. 7 shows a flowchart of a method for implanting a stimulation lead having a tissue in-growth anchor embodiment.

### DETAILED DESCRIPTION OF THE INVENTION

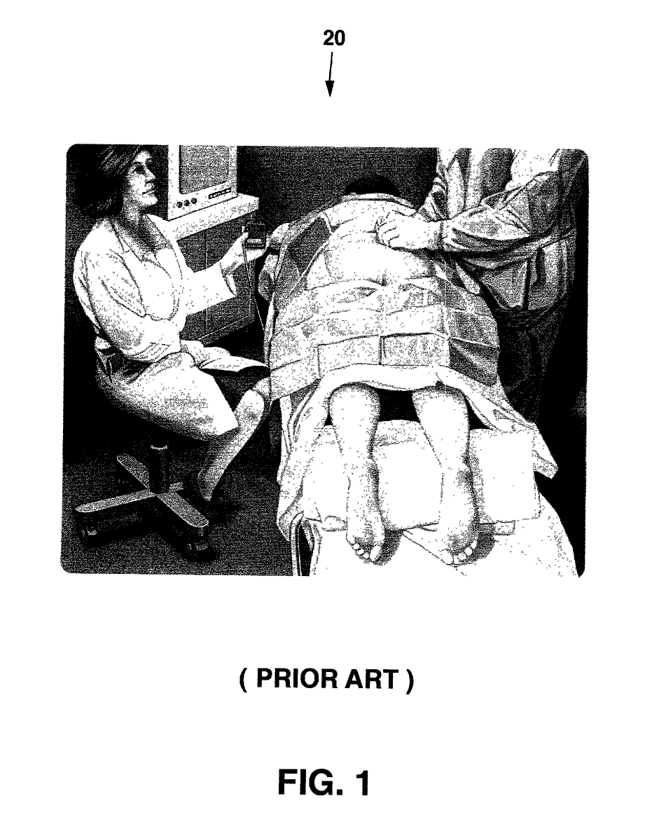

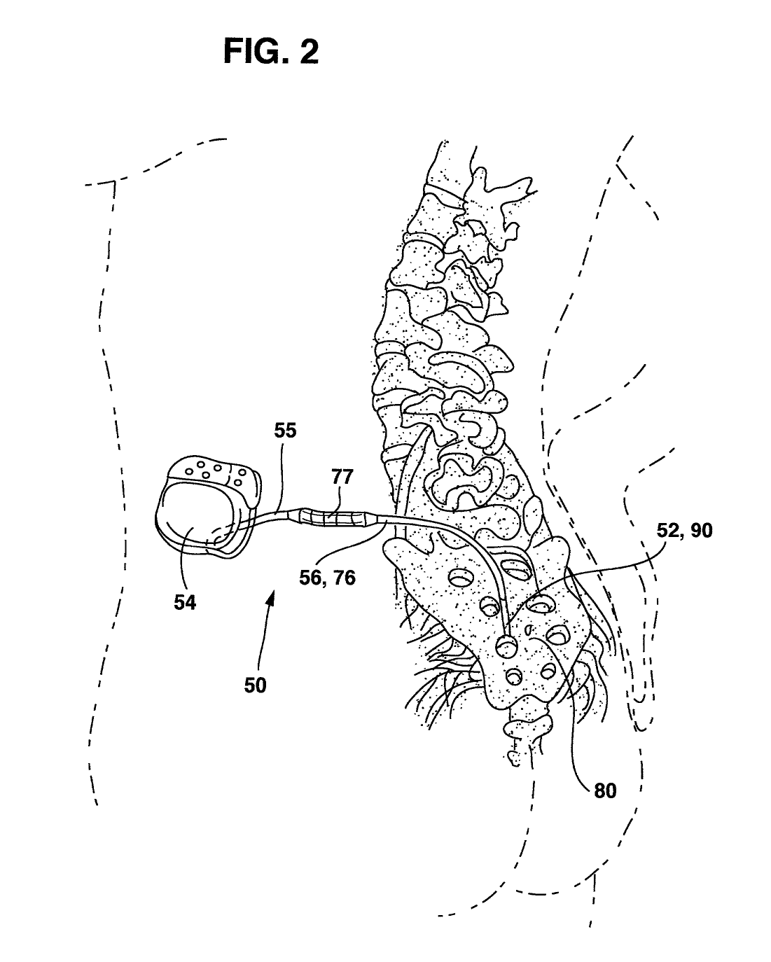

FIG. 1 (prior art) shows an environment of an patient undergoing a sacral stimulation procedure 20, and FIG. 2 shows a neurological stimulation system embodiment. The method for implanting a neurological stimulation lead 56 having an in-growth tissue anchor 52 can be performed in a wide variety of locations that have a sterile field and common medical instruments such as an operating room, surgery center. The method and its many embodiments are typically performed by a urologist, but can be performed by many clinicians trained in stimulation lead 56 implantation. The patient is placed under local or general anesthesia. With local anesthesia, the method can potentially be performed in a clinician's office for greater accessibility and reduced costs. A sacral stimulation lead 56 can be implanted for a variety a purposes such as to treat pelvic floor disorders. Pelvic floor disorders include urinary control disorders, fecal control disorders, sexual dysfunction, and pelvic pain. The sacral nerves are assessable through an entry point along an insertion path into a foramen to reach a desired location. A neurostimulation system 50 can include a stimulation lead 56, a lead extension 55, a trial stimulator (not shown), an implantable neurostimulator 54, a physician programmer (not shown), and a patient programmer (not shown).

An implantable neurological stimulation system 50 comprises an implantable neurological stimulator 54, at least one implantable stimulation lead 56, at least one electrode 60; and, an in-growth anchor 52. The implantable neurological stimulator 54 provides a programmable stimulation signal that is delivered to a desired location or target to stimulate selected nerves. The implantable neurological stimulator 54 is typically implanted in a subcutaneous pocket around the upper buttocks sometime after the stimulation lead 56 has been implanted and its effectiveness verified. The physician programmer is used by the clinician to communicate with the implantable neurological stimulator 54 to program the stimulation signal produced by the implantable neurological stimulator 54. The patient programmer allows the patient to communicate with the implantable neurological stimulator 54 to control certain parameters of the stimulation signal typically selected by a clinician. With a pelvic floor disorder, a patient can typically control stimulation signal parameters such as voltage amplitude. Neurostimulation systems 50 with the components discussed above are available from Medtronic, Inc. in Minneapolis, Minn.

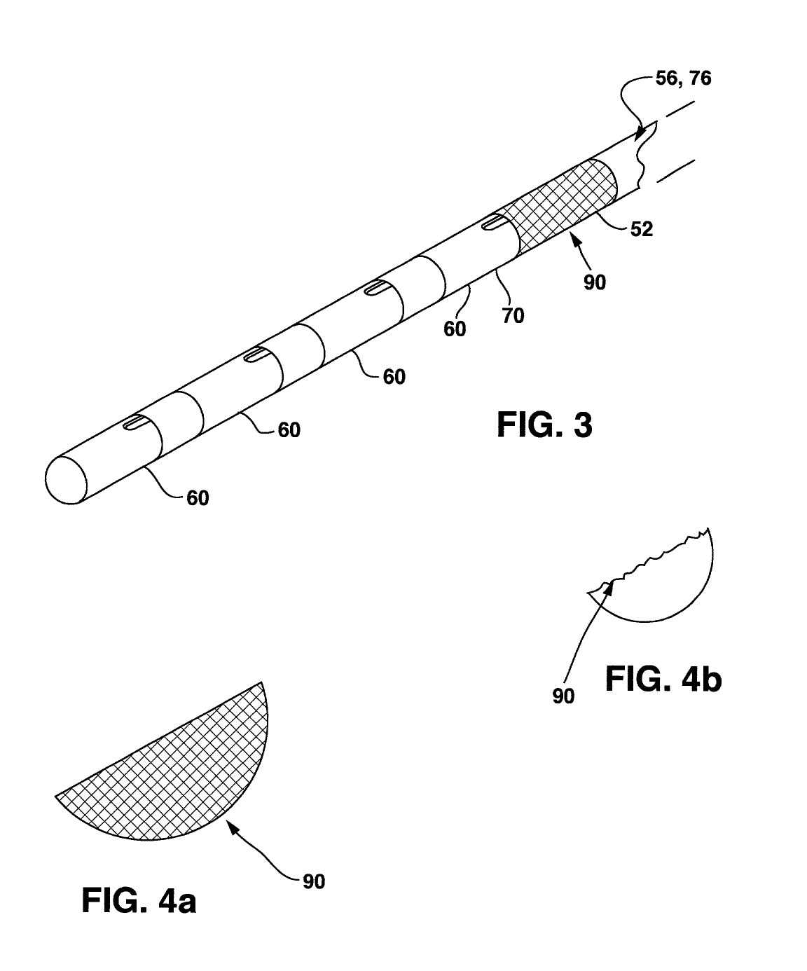

FIG. 3 shows a neurological stimulation lead 56 embodiment. The implantable stimulation lead 56 configured for percutaneous implantation has a lead body 76, at least one electrode 60, at least one connector 77, and at least one conductor. The lead body 76 has a distal body end, a proximal body end. The electrode 60 is coupled to the distal body end, and the connector 77 is coupled to the proximal body end. There is a conductor carried in the lead body 76 to electrically connect the electrode 60 to the connector 77. The conductor is insulated by the lead body 76. The implantable stimulation lead 56 can be a Medtronic Model 3886 quadrapolar lead having a diameter of approximately a 0.127 cm (0.050 inch) and designed to accept a stylet through the center of the stimulation lead 56 to assist in insertion.

FIG. 3 shows a neurological stimulation lead 56 embodiment, and FIGS. 4a-4b show in-growth anchor 52 material embodiments. The in-growth anchor 52 is positioned proximal to and not in contact with the electrode 60. The in-growth anchor 52 serves as a means for tissue in-growth coupleable to the stimulation lead 56 to receive tissue in-growth to anchor the stimulation lead 56 in a patient. The in-growth anchor 52 fixes the stimulation lead 56 to prevent the stimulation lead 56 from migrating away from the position selected by the implanting clinician. The in-growth anchor 52 is typically positioned in the range from about 5 millimeters (0.197 inches) to about 25 millimeters (0.984 inches) from the most proximal electrode 70. The tissue in-growth anchor 52 comprises a therapy attachment element and a tissue fixation element. The therapy attachment element couples the tissue in-growth anchor 52 to the implantable neurological stimulation lead 56. The tissue in growth anchor 52 configured for receiving tissue in-growth to anchor the implantable neurological stimulation lead 56. The tissue fixation element can be a three-dimensional matrix material with microvoids or pores 90 with sized in the range from about 1 micron to about 500 microns.

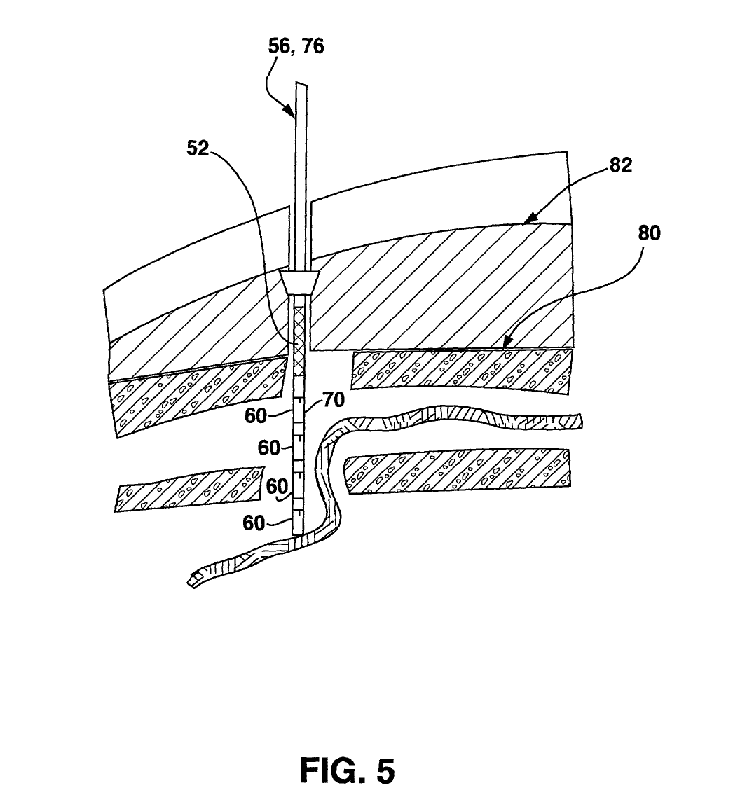

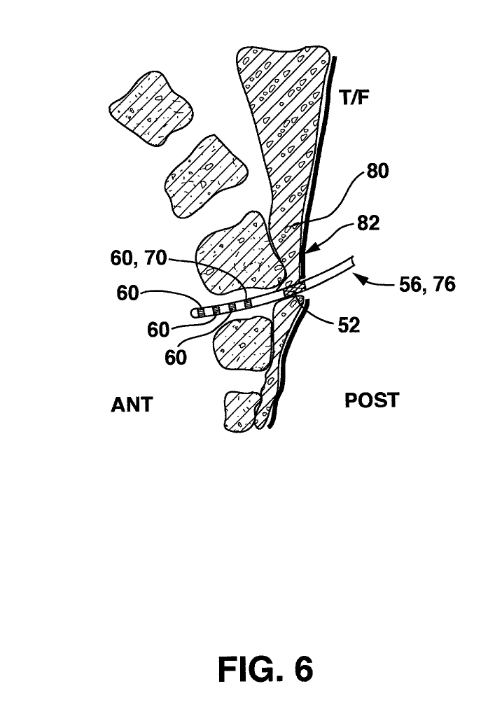

FIG. 5 shows a schematic of an implanted sacral stimulation lead 56 embodiment, and FIG. 6 shows an anatomical drawing of a sacrum embodiment. The in-growth anchor 52 is secured at a selected anchor position at least until sufficient tissue in-growth has anchored the tissue in-growth anchor 52 at a tissue location. The tissue fixation location can be near where the implantable neurological stimulation lead 56 enters the human body on subcutaneous tissue. The in-growth anchor 52 is positioned proximate to a sacrum posterior surface 80. The in-growth anchor 52 extends from about the sacrum posterior surface 80 in the posterior direction. The in-growth anchor 52 extends from about the sacrum posterior surface 80 in the posterior direction through a fascia layer 74. The in-growth anchor 52 extends from the sacrum posterior surface 80 in the range from about 5 millimeters (0.197 inches) to about 25 millimeters (0.984 inches).

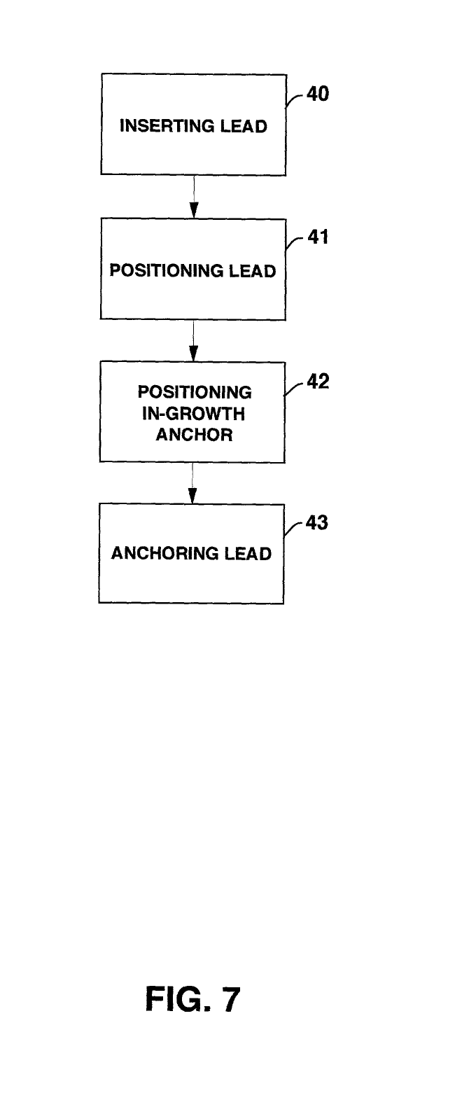

FIG. 7 shows a flowchart of a method for implanting a stimulation lead 56 having a tissue in-growth anchor 52 embodiment. The method for anchoring an implantable neurological stimulation lead 56 having a tissue in-growth anchor 52 comprises the following elements. The implantable neurological stimulation lead 56 having a tissue in-growth anchor 52 is inserted into a human body 40. The stimulation lead 56 is positioned at a selected delivery position in the human body 41. The tissue in-growth anchor 52 is positioned at a selected anchor position in the human body 42. The anchor position can be near where the stimulation lead 56 enters the human body on subcutaneous tissue. The tissue in-growth anchor 52 becomes anchored 43 once sufficient tissue in-growth has attached the tissue in-growth anchor 52 to tissue. The method can also include securing the in-growth anchor 52 with a mechanical fixation anchor at the selected anchor position at least until sufficient tissue in-growth has anchored the tissue in-growth anchor 52.

The method for anchoring an implantable neurological stimulation lead 56 having a tissue in-growth anchor 52 can be practiced as the following minimally invasive embodiment discussed referring to FIGS. 5-7. Prior to beginning the minimally invasive method embodiment for sacral electrical stimulation lead 56 implantation in a patient, the following preparatory actions are typically taken. A local anesthetic is typically applied to anesthetize the area where the stimulation lead 56 will be implanted such as posterior to the sacrum. Since embodiments of the method permit use of a local anesthetic, patients can be treated on an outpatient basis to greatly reduces costs over inpatient care and reduce recovery time. This significant cost reduction also makes sacral stimulation lead 56 implantation and its many beneficial therapies available to more patients because healthcare payers are more likely to cover procedure costs. Also by using local anesthesia, the implanting clinician can use the patient's conscious sensory response to stimuli such as trial stimulation to aid in placing the stimulation lead 56. By using the patient's conscious sensory response during stimulation lead 56 placement, the stimulation lead 56 can be more accurately placed reducing the potential for an ineffective therapy and reducing the potential for patient injury caused by a misplaced lead. Other forms of anesthesia can also be used such as general anesthesia. Once the patient has been anesthetized, the first method embodiment can begin.

A needle is inserted posterior to the sacrum through an entry point typically created with the needle. The needle can take a variety of forms such as a needle without a hub (cannula), a solid rod with a sharp tip, a needle with a hub that can be removed for example by a cutting tool, or a foramen needle modified to have an extended length and a hub that can be removed with a cutting tool. The entry point is typically a percutaneous entry created when the needle is inserted. The needle is hand guided into the foramen along an insertion path to a desired location. The foramen's approximate location can be found using anatomical landmarks, fluoroscopy, or x-rays. When guiding the needle, the position of the needle can be sensed by a variety of means such as by applying an electrical signal to the needle to evoke a patient response such as a motor or sensory response. Once the needle is in position, the needle can remain in the position to serve as a guide for the dilator, or in the alternative a guide wire can be inserted through the needle. When the needle is used as a guide for the dilator, the needle hub typically must be removed before the dilator can be guided over the needle. Alternatively, a guide wire can be used as the guide for the dilator. The guide wire can be a flexible guide wire, a stiff guide wire, or a stylet. Once the guide wire is in position, the needle can be removed, and the guide wire can serve as a guide for the dilator.

The insertion path is dilated with a dilator to a diameter sufficient for inserting a stimulation lead 56. The needle is removed from the insertion path, or alternatively the guide wire is removed from the insertion path. When removing the needle from the insertion path, care should be taken to avoid displacing the dilator. The stimulation lead 56 is inserted to the desired location. Since the chronic stimulation lead 56 is being inserted directly without the requirement for a separate test stimulation lead (not shown), such as a Medtronic Test Simulation Lead Model 3057, the chronic stimulation lead 56 can be placed without positioning repeatability variation. Also, there is a greater correlation between acute test stimulation and chronic therapy stimulation because the same lead is performing both test stimulation and therapy stimulation. The desired location can be any area of the sacrum intended to achieve a therapeutic effect such as into the foremen. One way to verify the stimulation lead's position is to apply an electrical signal to the stimulation lead to evoke a patient motor or sensory response. Other ways to verify the stimulation lead's position include imaging techniques such as fluoroscopy and x-ray. When inserting the implantable stimulation lead 56, the lead is advanced through the dilator to the desired location for stimulation. The dilator is removed from the insertion path. When removing the dilator from the insertion path, care should be taken to avoid displacing the stimulation lead 56. Additionally, stimulation lead 56 position should be re-verified by one of the previously discussed techniques. The tissue in-growth anchor 52 will begin fixing itself to surrounding tissue to anchor the stimulation lead 56.

Thus, embodiments of the implantable neurological stimulation lead 56 with tissue in-growth anchor 52 are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. An implantable neurological stimulation system having a tissue in-growth anchor, comprising:
an implantable neurostimulator;
at least one implantable stimulation lead coupled to the implantable neurostimulator;
at least one electrode coupled to the implantable stimulation lead; and,
an in-growth anchor coupleable to the implantable stimulation lead and configured for anchoring the implantable stimulation lead to tissue, the in-growth anchor positioned proximal to and not in contact with the electrode.

2. The implantable neurological stimulation system as in claim 1 wherein the in-growth anchor is positioned proximally to the most proximal electrode.

3. The implantable neurological stimulation system as in claim 2 wherein the in-growth anchor is positioned in the range from about 5 millimeters to about 25 millimeters from the most proximal electrode.

4. The implantable neurological stimulation system as in claim 1 wherein the tissue is located near where the implantable neurological stimulation lead enters the human body on subcutaneous tissue.

5. The implantable neurological stimulation system as in claim 1 wherein the in-growth anchor is configured to be positioned proximate to a sacrum posterior surface.

6. The implantable neurological stimulation system as in claim 5 wherein the in-growth anchor is configured to extend from about the sacrum posterior surface in the posterior direction.

7. The implantable neurological stimulation system as in claim 6 wherein the in-growth anchor is configured to extend from about the sacrum posterior surface in the posterior direction through a fascia layer.

8. The implantable neurological stimulation system as in claim 5 wherein the in-growth anchor is configured to extend from the sacrum posterior surface in the range from about 5 millimeters to about 25 millimeters.

9. The implantable neurological stimulation system as in claim 1 wherein the in-growth anchor is secured at a selected anchor position at least until sufficient tissue in-growth has anchored the tissue in-growth anchor at a tissue location.

10. An implantable neurological stimulation system having a tissue in-growth anchor, comprising:
an implantable neurostimulator;
at least one implantable stimulation lead coupled to the implantable neurostimulator;
at least one electrode coupled to the implantable stimulation lead and an implantable therapy delivery device; and,
means for tissue in-growth coupleable to the implantable stimulation lead, the means for in-growth configured to receive tissue in-growth to anchor the implantable stimulation lead to tissue.

11. An implantable neurological stimulation lead configured for percutaneous implantation having a tissue in-growth anchor, comprising:
a lead body having a distal body end, a proximal body end;
at least one electrode coupled to the distal body end;
at least one connector coupled to the proximal body end;
at least one conductor carried in the lead body, the conductor electrically connecting the at least one electrode to the at least one connector and insulated by the lead body;
at least one in-growth anchor coupleable to the implantable neurological stimulation lead and configured for anchoring the implantable neurological stimulation lead to tissue, the in-growth anchor positioned proximal to the electrode.

12. The implantable neurological stimulation lead as in claim 11 wherein the in-growth anchor is positioned proximally to the most proximal electrode.

13. The implantable neurological stimulation lead as in claim 12 wherein the in-growth anchor is positioned in the range from about 5 millimeters to about 25 millimeters from the most proximal electrode.

14. The implantable neurological stimulation lead as in claim 1 wherein the tissue is located near where the implantable neurological stimulation lead enters the human body on subcutaneous tissue.

15. The implantable neurological stimulation lead as in claim 1 wherein the in-growth anchor is configured to be positioned proximate to a sacrum posterior surface.

16. The implantable neurological stimulation lead as in claim 15 wherein the in-growth anchor is configured to extend from about the sacrum posterior surface in the posterior direction.

17. The implantable neurological stimulation lead as in claim 16 wherein the in-growth anchor is configured to extend from about the sacrum posterior surface in the posterior direction through a fascia layer.

18. The implantable neurological stimulation lead as in claim 15 wherein the in-growth anchor is configured to extend from the sacrum posterior surface in the range from about 5 millimeters to about 25 millimeters.

19. A tissue in-growth anchor for an implantable neurological stimulation lead, comprising:
a lead attachment element coupling for the tissue in-growth anchor to the implantable neurological stimulation lead; and,
a tissue fixation element connected to the implantable neurological stimulation lead, the tissue fixation element configured for receiving tissue in-growth to anchor the implantable neurological stimulation lead.

20. The implantable neurological stimulation lead as in claim 19 wherein the in-growth anchor is a textured material.

21. The implantable neurological stimulation lead as in claim 20 wherein the textured material is a three-dimensional matrix with microvoids in the range from about 1 micron to about 500 microns.

22. A method for anchoring an implantable neurological stimulation lead having a tissue in-growth anchor, comprising:
inserting an implantable neurological stimulation lead having a tissue in-growth anchor into a human body;
positioning the implantable neurological stimulation lead at a selected delivery position in the human body;
positioning the tissue in-growth anchor at a selected anchor position in the human body; and,
anchoring the tissue in-growth anchor once sufficient tissue in-growth has attached the tissue in-growth anchor to tissue.

23. The method as in claim 22 further comprising securing the in-growth anchor at the selected anchor position at least until sufficient tissue in-growth has anchored the tissue in-growth anchor.

24. The method as in claim 22 wherein the tissue location is near where the implantable neurological stimulation lead enters the human body on subcutaneous tissue.
